(19) United States
(12) Patent Application Publication
(10) Pub. No.: US 2005/0010178 A1
(43) Pub. Date: Jan. 13, 2005

(54) Expandable penetrating needle and method of use

(76) Inventor: Victor Katz, Columbus, OH (US)

Correspondence Address: STANDLEY LAW GROUP LLP, 495 METRO PLACE SOUTH, SUITE 210, DUBLIN, OH, 43017, US

(21) Appl. No.: 10/614,667

(22) Filed: Jul. 7, 2003

(51) Int. Cl. A61M 5/32

(52) U.S. Cl. 604/272

(57) ABSTRACT

An expandable penetrating needle of substantially conical shape when in an unexpanded state. The expandable penetrating needle has a needle shaft with a preferably sharp point at a first end, and a base at a second end. The base is adapted for attachment to a substantially hollow hub. The needle shaft is divided into separable segments. An additional device may be inserted through the hub and into the needle shaft. As the additional device is passed through the needle shaft, the segments thereof are caused to progressively separate, thereby causing a gradual expansion of an initial entryway produced by insertion of the point of the needle shaft into a material to be penetrated. The expandable penetrating needle may be used for medical as well as non-medical purposes.

### BACKGROUND AND SUMMARY OF THE INVENTION

The present invention relates to an expandable penetrating needle. More specifically, the present invention relates to a needle having a main body that is shaped essentially like a sharply pointed cone when in an unexpanded state, but that opens into a substantially cylindrical shape when expanded. The needle is substantially hollow, such that a second needle or other similar tubing may be passed therethrough. Insertion of a second needle or other tubing into the penetrating needle causes its expansion. The expandable penetrating needle may be used to pierce various materials, such as rubber or plastic. However, the expandable penetrating needle is especially well suited for use in venipuncture procedures, wherein the skin and vein of a patient must be penetrated.

Various procedures require the non-destructive piercing of a flexible or semi-rigid material, typically in order to gain access to the contents of a container to which the flexible material is affixed. Such materials are commonly formed from a plastic, or more commonly, from a rubber material. For example, rubber stoppers may be used to seal vials or other containers of laboratory chemicals, such as reagents and the like. Access and removal of such chemicals or other materials is typically accomplished by piercing the rubber stopper with a needle, cannula, or other similarly sharp-tipped hollow tool. The desired amount of chemicals may then be drawn into a syringe or other container. Upon removal of the needle or cannula, the hole produced in the stopper thereby is substantially resealed by the expansion of its rubber composition.

Another common procedure requiring the non-destructive piercing of a flexible material is the insertion of a needle into the tissue of a patient. One such procedure is commonly referred to as venipuncture, and involves passing a needle through a patients skin and into a vein, whereby access to the patients blood supply is accomplished. Venipuncture may be practiced as a portion of a variety of medical procedures. For example, drug introduction and the drawing of a blood sample (phlebotomy) both employ venipuncture. Various devices may be employed in a typical venipuncture procedure, such as, for example, a hypodermic needle and syringe, a single or multi-sample needle and an evacuated collection tube, and a winged (butterfly) needle that may be connected to an infusion set, a syringe, or an evacuated collection tube. Venipuncture is also practiced when inserting a catheter into a patient's vein. In this case, a needle is used to penetrate the vein and a catheter tube is thereafter inserted into the vein, over the needle, and into the hole in the vein made by the needle.

Needles come in various shapes and sizes, depending largely on their intended use. For example, needles may be single-ended, such as for use with a syringe, or double-ended, such as for use with an evacuated collection tube. Needles generally consist of a shaft that is inserted into the patient, and a hub for attachment to a syringe, collection tube, or other device. The penetrating end of the needle is typically beveled, with the leading edge of the bevel forming the point of the needle. Double-ended needles typically have a bevel and point at both ends. The point is typically made as sharp as possible to provide for penetration of a patient's skin and vein (and the rubber stopper of a collection tube in the case of double-ended needles). Needles are also generally hollow to allow for the delivery of fluids or to allow for the withdrawal of an amount of a patient's blood. The passageway that runs through the length of the needle is commonly referred to as the lumen. The diameter of the lumen determines the gauge, or size, of the needle. In addition to their use in venipuncture procedures, such needles may also be used to pierce other flexible materials, as described above.

The use of such needles is not without problems, however—whether they are used to pierce flexible materials, such as plastic and rubber, or in venipuncture procedures. In order to properly withdraw or inject fluids through a needle, the lumen must be of adequate diameter. Adequate lumen diameter is based, at least somewhat, on the type and viscosity of the material to be passed through the needle. Additionally, a needle will encounter at least some resistance during piercing of a patient's skin, a rubber stopper, or another material to be penetrated. As such, the needle must have sufficient strength and rigidity to prevent it from bending or breaking during insertion. Thus, in addition to the diameter of the lumen, the needle must also have a wall thickness sufficient to impart the necessary rigidity thereto. This wall thickness contributes to the overall diameter of the needle, which diameter must pass through the material to be penetrated.

Obviously, a needle of greater diameter will create a larger opening in the material it penetrates. Therefore, it is typically desired, particularly in venipuncture, to use the smallest needle possible. The use of a smaller needle will encounter less resistance from a patient's skin and vein (or another material to be penetrated), thereby generally producing less pain and resulting in less bleeding. However, the minimum diameter of a needle used in venipuncture is limited. For example, when dispensing intravenous drugs, the needle must be able to adequately pass the material into the patients vein. In a catheter procedure, the needle must be large enough to create a hole of sufficient size to accept the catheter. And, in phlebotomy, a lumen of too small a diameter can cause damage to red blood cells, leading to an unusable blood sample.

The bevel common to typical needles is intended to make insertion (penetration) of the needle easier. The bevel allows the diameter of the needle to be inserted incrementally—as opposed to the instantaneous insertion of the needle's diameter that would occur with a blunt (squared off) tip. The use of a bevel also has drawbacks, however. For example, it is known to be somewhat difficult to produce a bevel on needles, especially those of small diameter (gauge), while simultaneously maintaining a sharp point and a clean lumen opening. Also, while the bevel allows for incremental insertion of the needle diameter, it also requires that a greater length of needle be inserted before materials may be withdrawn or injected therethrough. The bevel also imparts a larger surface area to the cutting portion of the needle.

Thus, use of typical, known needles, can be problematic for a number of reasons. For example, when using such needles to penetrate rubber stoppers or similar other closures, the sharpened bevel of the needle often cuts or otherwise abrades away portions of the closure material. Therefore, holes are eventually formed through the closure that cannot be closed by the expansion of its composition. The closure material may also enter into and clog the needle during insertion. When used in venipuncture, such needles tend to cut a substantial hole into the patient's skin and vein. The greater the gauge of the needle, the larger the hole, and the more pain a patient must endure. Additionally, such needles may be very difficult to use properly on a patient with small, collapsed, or “rolling” veins. In such cases, it is easily possible to pass the tip of the needle through the entire vein, or to completely miss the vein.

The expandable penetrating needle of the present invention overcomes the aforementioned shortcomings of typical, known needles. In comparison to typical needles, which require that the full diameter of the needle cut through the material to be penetrated (such as the skin and vein of a patient), the expandable penetrating needle of the present invention is designed to produce a smaller initial entryway. Thus, penetration of a subject material by the expandable penetrating needle of the present invention is less invasive than penetration by typical needles.

In order to produce a minimal entryway through the material to be penetrated, the expandable penetrating needle of the present invention is preferably designed to have a substantially conical shape when in an unexpanded state. More specifically, the expandable penetrating needle has a shaft that is formed into a segmented cone having a sharp point. The shaft may be perforated, scored, or possess weakened sections, for example, that divide the cone into a plurality of substantially triangular sections. The base of the conical shaft may be attached to a variety of hubs or other similar attachment structures. For example, when employed in the medical field, the expandable penetrating needle may be designed for attachment to a hypodermic syringe, an evacuated collection tube holder, a butterfly infusion system, or a catheter insertion apparatus. A multitude of other configurations are also possible.

The segmented design of the expandable penetrating needle shaft allows it to expand upon insertion of another device. For example, a section of tubing, such as a second needle or catheter may be inserted into the hub of the expandable penetrating needle. As the second needle or section of tubing is moved toward the tip of the expandable penetrating needle, the individual segments of the conical shaft are forced to separate and expand. Expansion of the segments causes a stretching of the penetrated material surrounding the outside of the expandable penetrating needle shaft, thereby allowing the initially created entryway to be enlarged, and the second needle or tubing to pass therethrough. Consequently, it can be understood that the expandable penetrating needle of the present invention can be used to permit the substantially less invasive insertion of a device of considerably greater diameter than the entryway initially created by the needle. Because the expandable penetrating needle produces an enlarged entryway by gradual expansion, as opposed to cutting, penetration of a material can be achieved with less destructive effect on the penetrated material and, in the case of venipuncture, with less pain experienced by the patient.

As discussed above, an expandable penetrating needle of the present invention may have a plurality of uses, and may be connected to a variety of other devices. A better understanding of the expandable penetrating needle of the present invention can be gained by reference to the following detailed description of certain exemplary embodiments thereof.

### BRIEF DESCRIPTION OF THE DRAWINGS

In addition to the features mentioned above, other aspects of the present invention will be readily apparent from the following descriptions of the drawings and exemplary embodiments, wherein like reference numerals across the several views refer to identical or equivalent features, and wherein:

FIG. 1a is an enlarged view illustrating a known type of needle, commonly referred to as a hypodermic needle;

FIG. 1b is an enlarged view illustrating a similar, known needle, and its use in a catheter assembly;

FIG. 2a depicts a venipuncture procedure, wherein a typical hypodermic needle has been inserted into a normal vein of a patient;

FIG. 2b depicts a venipuncture procedure, wherein a typical hypodermic needle has been inserted into a small, or collapsed vein of a patient;

FIG. 3a is an enlarged view, in partial cross-section, showing one embodiment of an expandable penetrating needle of the present invention in an unexpanded state;

FIG. 3b shows the expandable penetrating needle of FIG. 3a in an expanded state;

FIG. 4a is an enlarged view showing another embodiment of an expandable penetrating needle of the present invention in an unexpanded state;

FIG. 4b shows the expandable penetrating needle of FIG. 4a in an expanded state;

FIG. 5a is an enlarged cross-sectional view of the expandable penetrating needle of FIG. 4a;

FIG. 5b is an enlarged cross-sectional view, showing a second needle partially inserted into the expandable penetrating needle of FIG. 5a;

FIG. 5c is an enlarged, partial cut-away view, showing the needle assembly of FIG. 5b with substantially full insertion of the second needle into the expandable penetrating needle;

FIG. 6a depicts a venipuncture procedure, wherein an expandable penetrating needle of the present invention has been inserted into a small, or collapsed vein of a patient, to facilitate the subsequent insertion of a typical hypodermic needle; and

FIG. 6b shows the arrangement of FIG. 6a, with the hypodermic needle fully inserted through the expandable penetrating needle and into the patient's vein.

### DETAILED DESCRIPTION OF THE EXEMPLARY EMBODIMENT(S)

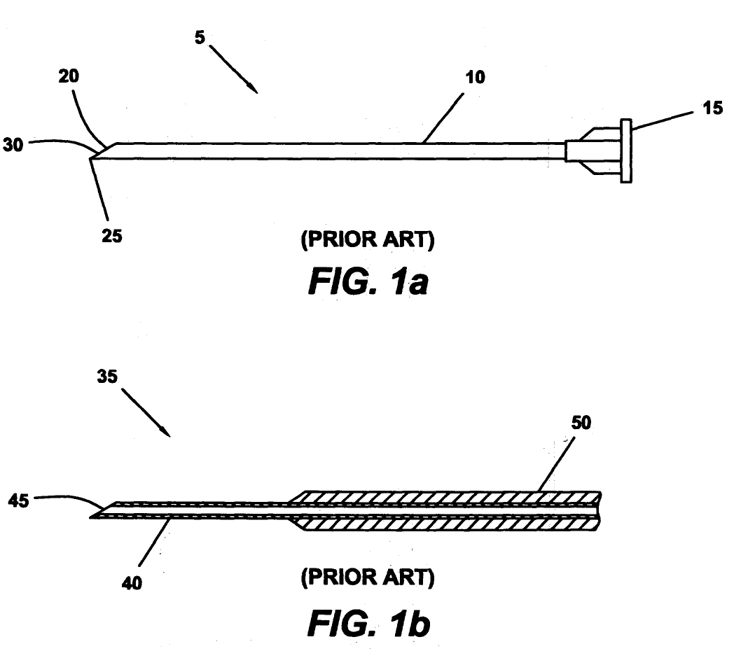

The expandable penetrating needle of the present invention is designed to be used in a variety of applications, including those in which typical needles are currently employed. A typical hypodermic needle 5 can be observed by reference to FIG. 1a. The hypodermic needle 5 can be seen to have a shaft 10 that is attached at a first end to a hub 15. The hub 15 is provided to attach or connect the needle 5 to another device, such as a syringe or evacuated collection tube holder, for example. The second end of the shaft 10 is provided with a bevel 20 that forms a point 25 at the tip of the needle 5. The bevel 20 acts as a cutting edge to allow the shaft 10 of the needle 5 to pass through a material to be penetrated. The hollow interior of the needle is generally referred to as the lumen 30. The diameter of the lumen 30 determines the gauge, or size, of the needle.

Other types of needles also exist. For example, double-ended needles, with each end having a bevel, are produced for use with evacuated collection tubes/holders. Such devices are commonly used during phlebotomy. Other needles may be used to provide an entryway, or pilot hole, for the insertion of another device. For example, in the medical field, needles are typically used to create an entryway into a patient's vein for the insertion of a catheter. A portion of such an assembly 35 is depicted in FIG. 1b. As can be seen from this portion of the catheter assembly 35, a penetrating needle 40 is located interior to a section of hollow catheter tubing 50. It is also possible for the catheter tubing to reside inside the lumen 45 of the needle, although such an arrangement is generally less common. In such an assembly, the needle 40 is first inserted through a patient's skin and into a vein. Once the needle is secured, the catheter tubing 50 is passed over the needle 40, and inserted into the vein through the hole therein made by the needle. As the catheter tubing 50 is generally of considerably larger diameter than the needle 40, the leading end of the catheter tubing may be beveled or otherwise designed to facilitate its entry into the vein. However, even with such treatment, forcing the catheter into the vein will generally cause discomfort to the patient, and may be harmful to the vein and other tissue of the patient through which it passes. Once the catheter tubing 50 is inserted, provision is generally made for removal of the needle 40.

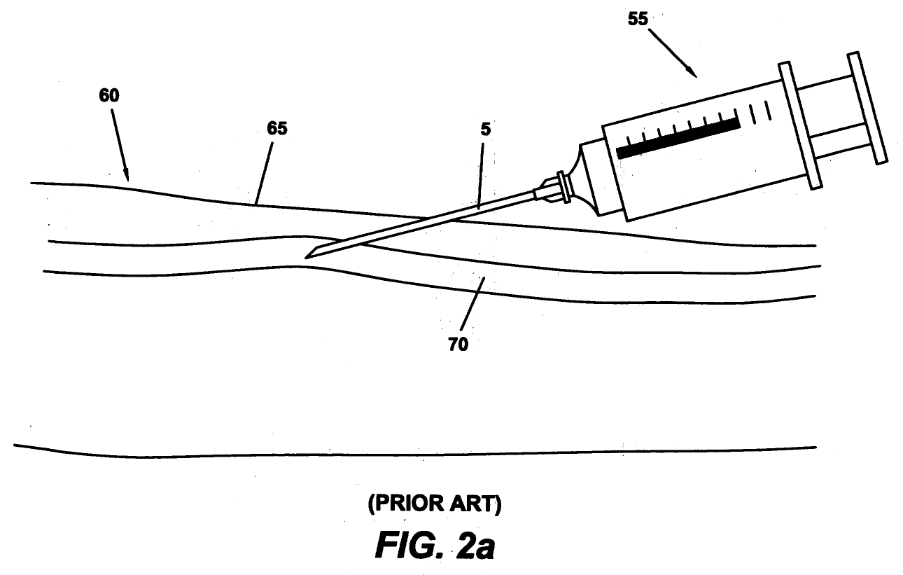

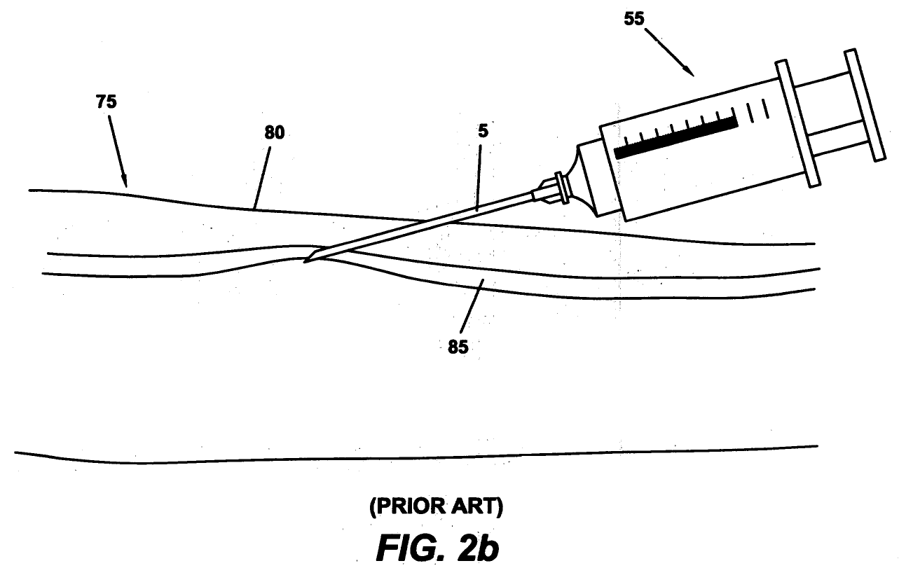

Use of a hypodermic needle, like that shown in FIG. 1a, can be seen by reference to FIGS. 2a and 2b. In FIG. 2a, the hypodermic needle 5 is shown attached to a syringe 55. The hypodermic needle 5 has been inserted into the arm 60 of a patient, whereby the needle has penetrated both the skin 65 and a vein 70 of the patient. The needle 5 and syringe 55 are shown to be oriented at a slight angle with respect to horizontal (and the patient's arm), which angle is typically about 15°. As can be observed from FIG. 2a, and the following example, an entryway is created through the tissue of the patient's arm by the needle 5, the angle of entry and beveled cutting edge 20 of which can increase the size of the entryway beyond that of the needle diameter. In this particular example, the needle 5 has been inserted into a substantially normal vein 70 (i.e., the vein is of normal size and is not collapsed).

The hypodermic needle 5 and syringe 55 of FIG. 2a are again shown inserted into the arm 75 of a patient in FIG. 2b. Again, the needle 5 and syringe 55 are shown to be oriented at a slight angle with respect to horizontal, and the needle has penetrated through the patient's skin 80 and into a vein 85. However, in this example the vein 85 is shown to be collapsed, such as may occur from, for example, repeated injection (needle penetration) of the same site. A similar problem may occur when performing venipuncture on a patient with small veins, such as a child or an elderly person. In either case, the vein generally has a significantly reduced diameter. As such, it may be exceedingly difficult, if not impossible, to insert the needle 5 into the vein without at least the tip 25 of the needle also penetrating partially or fully through both walls thereof. Further, insertion of the needle 5 to a point wherein less than the whole of the lumen 30 resides within the vein can cause blood loss and an incomplete blood draw or injection.

As can be understood from the above examples, when using penetrating needles, it is desirable to minimize the size of the entryway that must be cut through the material to be penetrated. Such is true whether the needle is used to pierce an inanimate object, such as a rubber or cork stopper, or the tissue of a living patient. In either case, minimizing the size of the cut entryway reduces damage to the material to be penetrated and allows the entryway to be more easily resealed after needle withdrawal. Additionally, as expressly shown in the example of FIG. 2b, it is also desirable to minimize the size (diameter and length) of such a needle when it is utilized to perform venipuncture on a collapsed, small, or rolling vein.

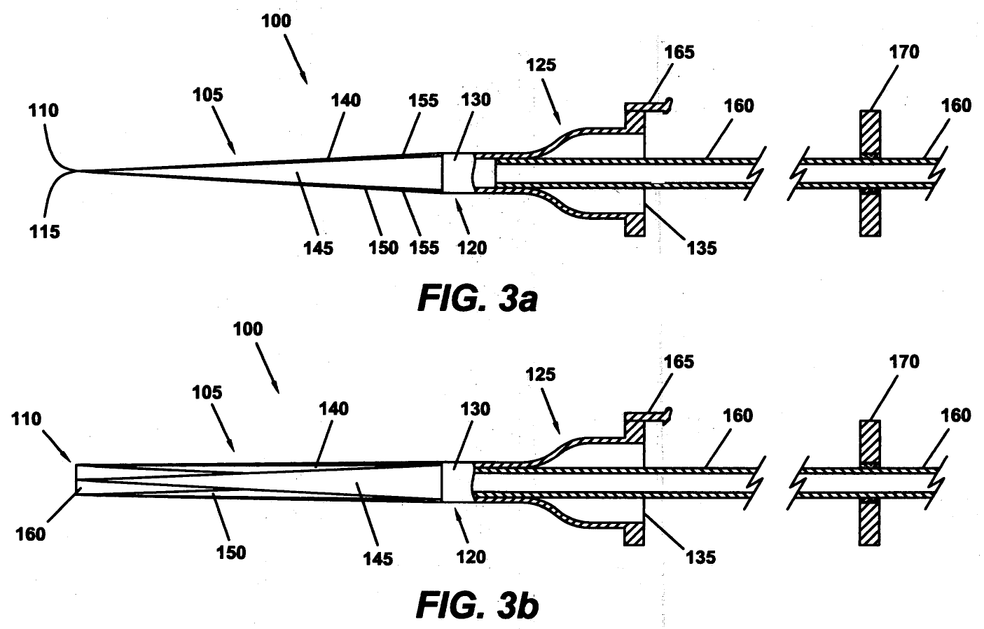

The expandable penetrating needle of the present invention minimizes or eliminates many of the disadvantages associated with the use of typical needles. One embodiment of an expandable penetrating needle 100 of the present invention can be observed in FIG. 3a. While the embodiment of the expandable penetrating needle shown in FIG. 3a, as well as the other exemplary embodiments depicted in the remaining drawing figures will be described specifically with respect to their use in venipuncture, it should be realized that such needles could also be used in a variety of other areas, such as has been briefly discussed above. The expandable penetrating needle 100 of FIG. 3a is shown in an unexpanded condition. The expandable penetrating needle 100 can be seen to have a shaft 105 of substantially conical shape. The distal end 110 of the shaft is formed into a sharp point (tip) 115 when the needle 100 is in an unexpanded state, while the proximal end (base) 120 of the shaft is attached to a hub 125. It is also contemplated that the base 125 of the needle shaft 105 could form the hub 125. The hub 125 has a forward portion 130 that is attached to the base 120 of the needle shaft 105, and an open, receiving end 135 opposite thereof. In this particular embodiment, the hub 125 is merely provided to be grasped by a user of the expandable penetrating needle 100 and to receive a section of tubing 160, such as catheter or intravenous tubing, that will eventually be passed through the needle. Thus, in this particular embodiment, it is not required that the hub 125 be of a specific shape or size.

When the expandable penetrating needle 100 will be used for phlebotomy, it is preferred that at least the forward portion 130 of the hub 125 be transparent, or otherwise allow a user of the needle to view a flow of blood from the patient's vein. In this manner, the phlebotomist is able to determine with certainty that the expandable penetrating needle 100 has been adequately inserted into the vein. As will be described in more detail below, the shaft 105 of the expandable penetrating needle 100 may also be modified to facilitate this process.

The shaft 105 of the expandable penetrating needle 100 is shown to be divided into a plurality of segments 140, 145, 150. In this particular embodiment the shaft is divided into three segments 140, 145, 150, but a fewer or greater number of segments are also possible. The segments 140, 145, 150 may be produced by cutting or scoring the shaft, for example. If the expandable penetrating needle 100 is of molded construction, it is also anticipated that the segments 140, 145, 150 may be produced by providing minute areas of weakened (thinner) material therebetween. The cuts, score lines, or other means of segmenting the shaft 105 may run through the tip 115 or, alternatively, may stop slightly short of the tip to help ensure that the segments are maintained in a tightly abutting relationship while the expandable penetrating needle 100 is in an unexpanded state.

As shown in FIG. 3a, it is anticipated that the tubing 160 may be partially inserted into the hub 125 of the expandable penetrating needle 100 prior to insertion of the expandable penetrating needle into a patient. For example, the expandable penetrating needle 100 and tubing 160 may be provided as a pre-assembled kit, or the components may be supplied separately and assembled prior to use. Preferably, the fit of the outer diameter of the tubing 160 and the inner diameter of the hub 125 or the base 120 of the needle shaft 105, depending on needle construction, provides a substantial seal and also prevents inadvertent movement of the tubing toward the tip 115 of the expandable penetrating needle 100. Other means of tubing retention may also be employed. For example, it is contemplated that a threaded collar (not shown) may be provided at the open end 135 of the hub 125 to engage a like-threaded portion (not shown) of tubing. In this embodiment, rotation of the collar in a first direction controllably and incrementally forces the tubing into the needle, while rotation of the collar in an opposite direction withdraws the tubing from the needle. The threaded portion of the tubing may be integral thereto or, alternatively, may be applied to the tubing, such as in the form of an adhesively attachable section.

Once the needle tip 115 has been properly inserted into a patient's vein, the tubing 160 may be pushed through the needle shaft 105. The expandable penetrating needle 100 may be held in place by the user during this operation but, preferably, the needle is secured to the patient, such as with tape or the like. As the tubing 160 is pushed into the needle, the segments 140, 145, 150 are forced to progressively separate, causing the outside diameter of the needle shaft 105 to gradually increase, and thereby slowly stretching the initial entryway produced in the vein by the needle tip 115. As the tubing approaches the now open distal end 110 of the expandable penetrating needle 100, the needle shaft 105 becomes substantially cylindrical in shape, with its inner diameter typically, but not essentially, similar in dimension to the outside diameter of the tubing 160. A mark or similar indicator may be provided on the tubing 160 to alert the user when the distal end thereof has reached the distal end 110 of the needle shaft 105.

Once the tubing 160 has been properly inserted into the vein, the user has the option of retracting the expandable penetrating needle 100 from the patient. For example, if the tubing 160 will be used only for a short-term procedure, the user may simply leave the expandable penetrating needle 100 in its inserted position, whereafter it may be withdrawn from the patient along with the tubing. However, if the tubing 160 will be used for a long-term. procedure, the user may wish to remove the expandable penetrating needle 100 from the patient and allow only the tubing to remain in the vein. The expanded inner diameter of the needle shaft 105 allows the expandable penetrating needle 100 to be retracted from the patient and moved to a position along the tubing 160 that is safely removed from the patient and/or contact by the user or others. The retracted expandable penetrating needle 100 may be affixed to the tubing 160 by a number of means,.such as, for example, a piece of tape. However, it is contemplated that the hub 125 of the expandable penetrating needle 100 may also have an optional retention mechanism 165, such as the spring-loaded hook shown attached thereto. In this embodiment, the hook 165 is designed to mate with a corresponding connector 170 that is affixed to the tubing 160 at a location away from the patient. As with the expandable penetrating needle 100, the connector 170 may come pre-installed to the tubing 160, or may be installed to the tubing by the user prior to installation of the expandable penetrating needle. Preferably, the connector 170 is designed to be moveable along the length of the tubing 160, but to also firmly resist inadvertent movement. Such may be accomplished, for example, by carefully controlling the fit of the connector inner diameter to the outer diameter of the tubing 160, or by forming at least the inner diameter of the connector 170 from a material that firmly but releasably grips the tubing. Such constructions are known and need not be discussed in detail herein.

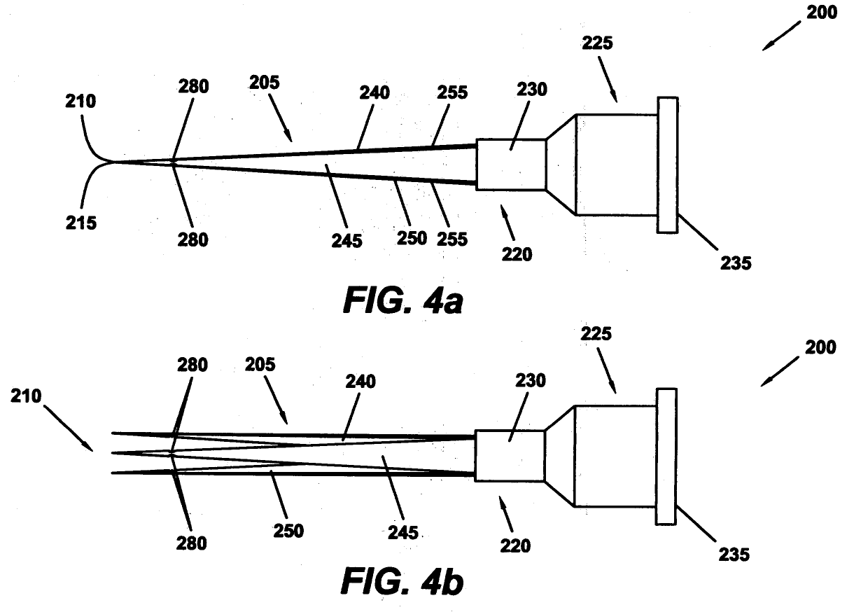

An alternate embodiment of an expandable penetrating needle 200 of the present invention in an unexpanded and expanded state is shown in FIGS. 4a and 4b, respectively. This embodiment of the expandable penetrating needle 200 is similar in design to the embodiment 100 shown in FIGS. 3a-3b. Like the embodiment of FIGS. 3a-3b, the expandable penetrating needle 200 can be seen to have a segmented shaft 205 of substantially conical shape with a distal end 210 thereof formed into a sharp point (tip) 215. The proximal end (base) 220 of the shaft 205 is attached to a specialized hub 225. The hub 225 has a forward portion 230 that is attached to the base 220 of the needle shaft 205, and an open, receiving end 235 opposite thereof. In this particular embodiment of the expandable penetrating needle 200, the hub 225 is specifically designed to mate with the hub of another needle, such as, for example, a hypodermic needle, or a double-ended needle. In this manner, the expandable penetrating needle 200 can be used in conjunction with another needle during venipuncture to more easily penetrate a patient's skin and vein. A hypodermic needle attached to a syringe or a double-ended needle attached to an evacuated collection tube/holder, for example, may thereafter be inserted through the expandable penetrating needle 200.

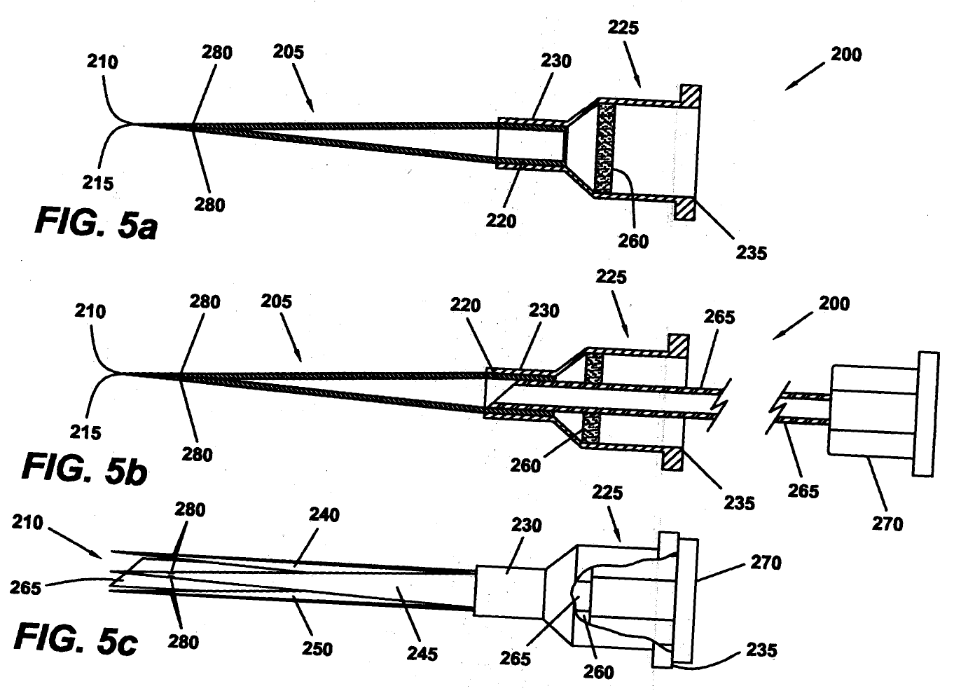

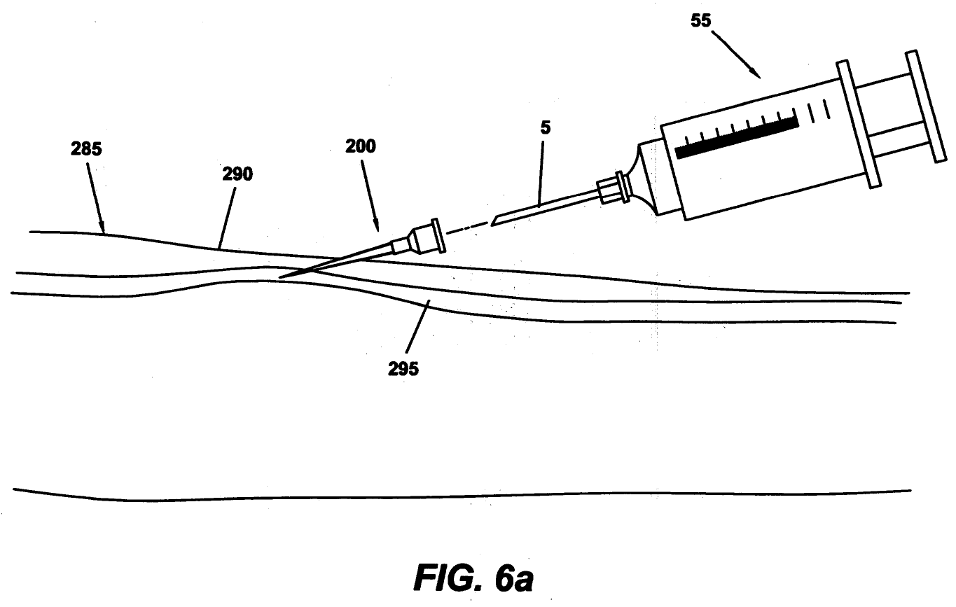

The expandable penetrating needle. 200 depicted in FIGS. 4a-4b can be seen in cross-section in FIGS. 5a-5c. As shown in FIG. 5a, the base 220 of the needle shaft 205 may be somewhat elongated to help in guiding the device, in this case the needle, that will be passed therethrough. In this particular embodiment of the expandable penetrating needle 200, the needle shaft 205 and hub 225 are of two-piece construction. Thus, the base 220 of the needle shaft 205 is shown to reside within a forward portion 230 of the hub 225. It is also contemplated that the forward portion 230 of the hub 225 could reside within the base 220 of the needle shaft 205. It is also possible that the expandable penetrating needle 200 could be of one piece construction, wherein the base 220 of the needle shaft 205 and the forward portion 230 of the hub 225 could have substantially the same inside diameter.

Like the shaft 105 of the expanding penetrating needle 100 shown in FIGS. 3a-3b, the shaft 205 of the expandable penetrating needle 200 is divided into three segments 240, 245, 250. The shaft 205 could also be divided into a fewer or greater number of segments. The segments 240, 245, 250 may be produced by cutting or scoring the shaft, for example. If the expandable penetrating needle 200 is of molded construction, it is also anticipated that the segments 240, 245, 250 may be produced by providing minute areas of weakened (thinner) material therebetween. The cuts, score lines, or other means of segmenting the shaft 205 may run through the tip 215 or, alternatively, may stop slightly short of the tip to help ensure that the segments 240, 245, 250 are maintained in a tightly abutting relationship while the expandable penetrating needle 200 is in an unexpanded state.

As shown in FIGS. 5a-5b, it is also possible that the hub 225 may contain a seal 260. The seal 260 is provided primarily for use in phlebotomy applications, to help prevent any of the initial flow of blood that occurs after insertion of the tip 215 of the expandable penetrating needle 200 into a patient's vein from exiting the open end 235 of the hub 225. The design of the seal 260 may depend on the type of second needle 265 to be passed through the expandable penetrating needle 200. For example, if a blunt-ended second needle is to be used, the seal may be substantially a grommet, wherein a predefined (but substantially sealed) passageway is located therethrough. If the second needle has a beveled end, it may be possible to employ a solid seal 260, wherein the second needle simply punctures the seal upon its insertion into the expandable penetrating needle 200.

As can be best observed by specific reference to FIG. 5b, when the second needle 265 is initially inserted into the expandable penetrating needle 200, it may be guided by the inside diameter of the hub 225, or in this particular case, the inside diameter of the base 220 of the needle shaft 205. The length of the guiding section may vary. It is also possible that the outside diameter of the second needle 265 may be smaller than the inside diameter of the hub 225 or base 220 of the needle shaft 205, in which case the second needle may not make contact with the expandable penetrating needle 200 until the second needle reaches the conical (tapered) portion of the needle shaft 205. In any event, as the second needle 265 is inserted further into the expandable penetrating needle 200, the shaft 205 thereof is caused to expand by a separation of the segments 240, 245, 250. The expandable penetrating needle 200 is shown in an expanded state in FIG. 5c.

As shown in FIG. 5c, this particular embodiment of the expandable penetrating needle 200 is designed to mate with, or engage, a hub 270 of the second needle 265. Further, in this particular embodiment, full insertion of the second needle 265 (as shown) into the expandable penetrating needle 200 is indicated by full insertion of the hub 270 of the second needle 265 into the hub 225 of the expandable penetrating needle 200. In this case, when the second hub 270 is fully inserted into the expandable penetrating needle hub 225, the tip 275 of the second needle 265 is substantially aligned with the distal end 210 of the expandable penetrating needle shaft 205. At full insertion, it is also possible that the tip 275 of the second needle 265 may reside slightly within the expandable penetrating needle shaft 205, or protrude slightly therefrom. However, it is preferred that the tip 275 the second needle 265 not be allowed to protrude too far from the distal end 210 of the expandable penetrating needle shaft 205, as it may penetrate through the bottom wall of the vein. In other embodiments, the hub 270 of the second needle 265 may not be inserted into the hub 225 of the expandable penetrating needle 200. For example, the distal end of the hub 270 of the second needle 265 may simply abut the open end 235 of the expandable penetrating needle 200. Alternatively, the hubs 225, 270 may not make contact due to, for example, the length of the second needle 265. In this case, it is preferable that the second needle 265 be marked to indicate the point of insertion wherein the tip 275 thereof will exit the open distal end 210 of the expandable penetrating needle shaft 205.

As mentioned above with respect to the embodiment of FIGS. 3a-3b, when used for phlebotomy, it is preferred that at least the forward portion 230 of the hub 225 be transparent, or otherwise allow a user of the expandable penetrating needle 200 to view a flow of blood from the patient's vein. In this manner, the phlebotomist is able to determine with certainty that the expandable penetrating needle 200 has been adequately inserted into the vein. As is best observed in FIGS. 4a-4b, the shaft 205 of the expandable penetrating needle 200 has also been modified to facilitate this process. More specifically, in this embodiment of the expandable penetrating needle, arcuate sections of the needle shaft segments 240, 245, 250 have been removed along the abutting edges 255 (i.e., along the score lines, cut lines, etc., that separate the segments) thereof. Thus, small elliptical-shaped apertures 280 are formed through the needle shaft 205 to allow a small amount of blood to flow into the interior of the expandable penetrating needle 200 while it is still in an unexpanded state. Preferably, the apertures 280 are located toward the tip 215 of the needle shaft 205 so that the initial blood flow may be established with only a minimal insertion of the needle tip into the patient's vein. It is further contemplated that more than one aperture like that shown may be located between the segments, or that one or more apertures of other shape and size may be located between the segments 240, 245, 250 of the needle shaft 200. Alternatively, a single aperture, or multiple apertures may be located wholly in each segment 240, 245, 250 of the needle shaft 205, as opposed to along the abutting edges 255 thereof.

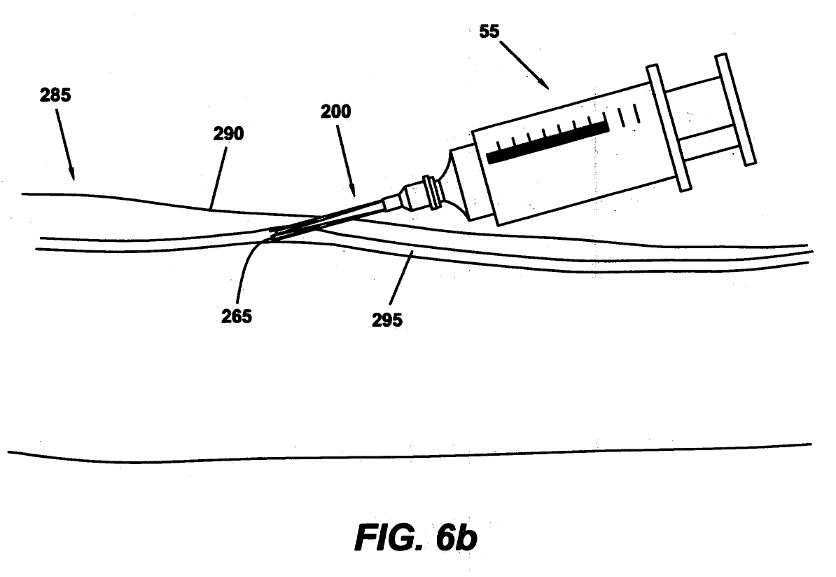

The expandable penetrating needle 200 of FIGS. 4a-4b and 5a-5c is shown in FIGS. 6a-6b during its use in a venipuncture procedure. In this example, the expandable penetrating needle 200 is used in conjunction with the hypodermic needle 5 and syringe 55 of FIGS. 2a-2b, although it should be realized that the expandable penetrating needle could be configured to work with virtually any venipuncture device. In the example of FIGS. 6a-6b, the hypodermic needle 5 and syringe 55 may be used, for example, to introduce an intravenous drug to the patient, or to obtain a blood sample.

In FIG. 6a, the expandable penetrating needle 200 has been inserted into the arm 285 of a patient, whereby the shaft 205 of the expandable penetrating needle has penetrated the patient's skin 290 and the tip 215 of the expandable penetrating needle has penetrated a vein 295 of the patient. In this example, the vein 295 is shown to be collapsed or of small size in order to demonstrate how an expandable penetrating needle of the present invention can facilitate entry thereto more easily than a typical needle. As can be seen, the expandable penetrating needle 200 is oriented at a slight angle with respect to horizontal (and the patient's arm). However, unlike with a typical needle, it should be understood that the conical shape of the expandable penetrating needle 200 may also allow an entry angle (not shown) into the patient's arm that is more vertical.

As can be observed from FIG. 6a, the tip 215 of the expandable penetrating needle 200 creates an initial entryway into the vein 295 of the patient. However, this initial entryway is of much smaller diameter than would be created by the cutting action of a typical beveled needle. Preferably, apertures 280 or other similar features in the needle shaft 205 allow an initial blood flow to be established, thereby indicating to the user thereof that proper entry of the expandable penetrating needle 200 into the vein 295 has been made. Once the expandable penetrating needle 200 has been properly inserted into the vein 295, it is preferably, but not necessarily, secured to the patient, such as with a piece of tape.

Once proper insertion of the expandable penetrating needle 200 into the vein has been established, the hypodermic needle 5 can be inserted therethrough. Progressive insertion of the hypodermic needle 5 into the expandable penetrating needle 200 causes a gradual expansion of the expandable penetrating needle shaft 205, as well as a gentle expansion of the initial entryway created in the vein 295. As can be seen in FIG. 6b, once the hypodermic needle 5 is substantially fully inserted into the expandable penetrating needle 200, the expandable penetrating needle shaft 205 becomes substantially fully open (expanded). Thus, the initial entryway in the vein 295 is stretched sufficiently to allow insertion of the larger diameter hypodermic needle 5, without forcing the hypodermic needle into the vein or requiring any additional cutting thereof. As can be seen in FIG. 6b, the expanding action of the needle shaft 205 acts to open up the previously collapsed section of the vein 295, thereby permitting access by the hypodermic needle 5 without fully penetrating or otherwise causing additional damage to the vein.

As a byproduct of the design of the expandable penetrating needle of the present invention, the expandable penetrating needle may be provided with a self-blunting feature. For example, in the embodiment shown in FIGS. 3a-3b, extension of the tubing 160 past the distal end 110 of the needle shaft 105 effectively blunts the end of the expandable penetrating needle 100. As the expandable penetrating needle 100 may be moved along the tubing 160 in a direction away from the patient after insertion of the tubing, or may otherwise be removed from the patient along with the tubing, accidental needle sticks can be prevented. Similarly, in the embodiment of the expandable penetrating needle 200 illustrated in FIGS. 4a-4b and 5a-5c, a blunt-ended needle may be substituted for the beveled second needle 265 shown. Consequently, insertion of a blunt-ended second needle to a point wherein its blunt end extends slightly beyond the end 210 of the shaft 205 of the expandable penetrating needle 200, acts as a safety device (guard) to prevent accidental needle sticks from the sharp points 215 thereof. Thus, in certain embodiments, the expandable penetrating needle of the present invention may be a part of a self-blunting apparatus.

While certain exemplary embodiments of the present invention are described in detail above, it should be realized that a pluarality of modifications are possible within the scope of the invention. For example, as mentioned above, the expandable penetrating needle of the present invention can be configured to connect to substantially any venipuncture device available. The expandable penetrating needle may be manufactured from virtually any material that provides the necessary strength and allows for the segmentation thereof. A multitude of different connectors, guards, etc., may be provided for storing and/or covering the expandable penetrating needle after its initial use. Further, the expandable penetrating needle of the present invention is not limited to use in venipuncture procedures, or to medical procedures in general but, rather, may be employed anywhere a needle would typically be used to pierce or penetrate a material. Therefore, the present invention is not to be considered limited by the above disclosure of exemplary embodiments, and modifications are possible without departing from the spirit of the invention as evidenced by the following claims:

1. An expandable penetrating needle comprising;
a needle shaft having a substantially conical shape when in an unexpanded state, said needle shaft having a tip at its distal end and a base at its proximal end; and
a substantially hollow hub having a forward end at said base of said needle shaft and a second, open end;
wherein said needle shaft is divided into a plurality of segments, the length of which extend substantially from said base portion to substantially said tip portion; and
wherein said open end of said hub is designed to allow at least a portion of an additional device of substantially cylindrical shape to be inserted through said needle shaft;
whereby, as said additional device is inserted through said needle shaft, said segments thereof. gradually separate, thereby causing an expansion of said needle shaft that allows said additional device to pass therethrough.

2. The expandable penetrating needle of claim 1, wherein at least a portion of said base of said needle resides within said forward end of said hub.

3. The expandable penetrating needle of claim 1, wherein at least a portion of said forward end of said hub resides within said base of said needle.

4. The expandable penetrating needle of claim 1, wherein said needle shaft and at least said forward end of said hub are of one-piece construction.

5. The expandable penetrating needle of claim 1, wherein said open end of said hub is adapted to mate with a portion of another device.

6. The expandable penetrating needle of claim 5, wherein said device is selected from the group consisting of a hypodermic needle hub, a hypodermic needle syringe, an evacuated collection tube needle hub, an evacuated collection tube holder, a catheter container hub, and a butterfly catheter needle hub.

7. The expandable penetrating needle of claim 1, wherein said segments extend through said-tip.

8. The expandable penetrating needle of claim 1, wherein said segments terminate prior to said tip.

9. The expandable penetrating needle of claim 1, further comprising one or more apertures passing through said needle shaft along abutting edges of said segments thereof.

10. The expandable penetrating needle of claim 1, further comprising one or more apertures passing through one or more of said segments of said needle shaft.

11. The expandable penetrating needle of claim 1, wherein said additional device is selected from the group consisting of plastic tubing, a hypodermic needle, an evacuated collection tube needle, a catheter needle, a catheter, and an IV needle.

12. The expandable penetrating needle of claim 1, wherein at least a portion of said forward end of said hub permits viewing of a material passing therethrough.

13. The expandable penetrating needle of claim 1, further comprising a retention device for securing said expandable penetrating needle to said additional device of substantially cylindrical shape or to another device attached thereto.

14. The expandable penetrating needle of claim 1, wherein said hub is a part of said needle shaft.

15. The expandable penetrating needle of claim 1, wherein said hub is attached to said needle shaft.

16. An expandable penetrating needle for use in a venipuncture procedure, comprising:
a needle shaft divided into a plurality of separable segments and having a substantially conical shape when in an unexpanded state, said needle shaft forming a point at its distal end for penetrating a vein and forming a base at its proximal end for connection to a hub;
a substantially hollow hub of some length having a forward end connected to said base of said needle shaft and a second, open end, disposed substantially opposite said forward end; and
at least one aperture passing through said needle shaft near said distal end thereof, said at least one aperture for facilitating an initial flow of blood into said needle shaft while said needle shaft is in an unexpanded state;
wherein said design of said expandable penetrating needle permits at least a portion of an additional device of substantially cylindrical cross-section to be passed through said needle shaft after said point of said needle shaft is located in said vein; and
wherein said segments of said needle shaft are adapted to gradually separate as said additional device of substantially cylindrical cross-section is progressively inserted through said needle shaft, thereby causing a simultaneous expansion of both said needle shaft and an initial entryway into said vein produced by said point thereof.

17. The expandable penetrating needle of claim 16, wherein at least a portion of said base of said needle resides within said forward end of said hub.

18. The expandable penetrating needle of claim 16, wherein at least a portion of said forward end of said hub resides within said base of said needle.

19. The expandable penetrating needle of claim 16, wherein said needle shaft and at least said forward end of said hub are of one-piece construction.

20. The expandable penetrating needle of claim 16, wherein said open end of said hub is adapted to mate with a portion of another device.

21. The expandable penetrating needle of claim 20, wherein said device is selected from the group consisting of a hypodermic needle hub, a hypodermic needle syringe, an evacuated collection tube needle hub, an evacuated collection tube holder, a catheter container hub, and a butterfly catheter needle hub.

22. The expandable penetrating needle of claim 16, wherein said segments extend through said point.

23. The expandable penetrating needle of claim 16, wherein said segments terminate prior to said point.

24. The expandable penetrating needle of claim 16, wherein said at least one aperture is located along an abutting edge of said needle shaft segments.

25. The expandable penetrating needle of claim 16, wherein said at least one aperture passes through one or more of said segments of said needle shaft.

26. The expandable penetrating needle of claim 16, wherein said additional device of substantially cylindrical cross-section is selected from the group consisting of plastic tubing, a hypodermic needle, an evacuated collection tube needle, a multi-use needle, a catheter needle, an IV needle, and a catheter.

27. The expandable penetrating needle of claim 16, wherein at least a portion of said forward end of said hub permits viewing of a material passing therethrough.

28. The expandable penetrating needle of claim 16, further comprising a seal located in said hub, said seal for preventing blood entering said unexpanded needle shaft through said at least one aperture from exiting said open end of said hub.

29. The expandable penetrating needle of claim 16, wherein said hub and/or said needle shaft are adapted to retain said expandable penetrating needle on said additional device of substantially cylindrical cross-section during insertion of said point into said vein.

30. The expandable penetrating needle of claim 16, further comprising a retention device for securing said expandable penetrating needle to said additional device of substantially cylindrical cross-section or to another device attached thereto.

31. The expandable penetrating needle of claim 16, further comprising a threaded collar rotatably attached to said hub near the open end thereof to engage a like-threaded portion of a section of tubing, rotation of said collar causing controlled movement of said tubing into or away from said needle shaft.

32. A method of performing venipuncture, comprising:
providing an expandable penetrating needle, said expandable penetrating needle comprising:
(a) a needle shaft divided into a plurality of separable segments and having a substantially conical shape when in an unexpanded state, said needle shaft forming a point at its distal end for penetrating a vein and forming a base at its proximal end for connection to a hub;
(b) a substantially hollow hub of some length having a forward end connected to said base of said needle shaft and a second, open end, disposed substantially opposite said forward end; and
(c) at least one aperture passing through said needle shaft near said distal end thereof, said at least one aperture for facilitating an initial flow of blood into said needle shaft while said needle shaft is in an unexpanded state;
installing said expandable penetrating needle to a patient, such that said point of said needle shaft properly penetrates a vein of said patient, thereby causing an initial entryway therein;
while securing said expandable penetrating needle, inserting an end of an additional venipuncture device through said open end of said hub and into said needle shaft; and
urging said end of said additional venipuncture device to pass through said needle shaft;
whereby progressive movement of said end of said additional venipuncture device through said needle shaft causes a gradual separation of said needle shaft segments, thereby causing said initial entryway into said vein to expand to a size that allows for substantially unimpeded passage of said end of said additional venipuncture device therethrough.

33. The method of claim 32, wherein said additional venipuncture device is selected from the group consisting of plastic tubing, a hypodermic needle, an evacuated collection tube needle, a multi-use needle, a catheter needle, an IV needle, and a catheter.

34. The method of claim 32, further comprising, after insertion of said additional venipuncture device into said vein, withdrawing said expandable penetrating needle from said patient and along said additional venipuncture device, and securing said expandable penetrating needle to said additional venipuncture device.
